(19) United States
(12) Patent Application Publication
(10) Pub. No.: US 2026/0166299 A1
(43) Pub. Date: Jun. 18, 2026

(54) CIRCULATION SUPPORT DEVICES, SYSTEMS, AND METHODS

(71) Applicant: Boston Scientific Scimed, Inc., Maple Grove, MN (US)

(72) Inventors: Brice Lee Shireman, Maple Grove, MN (US); Jeffrey Matthew Lucas, Eden Prairie, MN (US); Reggie Roth, Monticello, MN (US)

(73) Assignee: Boston Scientific Scimed, Inc., Maple Grove, MN (US)

(21) Appl. No.: 19/421,663

(22) Filed: Dec. 16, 2025

Related U.S. Application Data
(60) Provisional application No. 63/734,455, filed on Dec. 16, 2024.

(51) Int. Cl. A61M 60/17; A61M 60/216; A61M 60/411; A61M 60/81; A61M 60/90

(52) U.S. Cl. CPC A61M 60/17; A61M 60/216; A61M 60/411; A61M 60/81; A61M 60/90; A61M 2205/0266; A61M 2210/125

(57) ABSTRACT

A mechanical circulatory support system may include a blood pump and a cannula extending distally from the blood pump. The blood pump may include a motor and an impeller assembly in communication with the motor. The impeller assembly may be configured to pump blood from a left ventricle of a heart to an aorta in response to actuation of the motor. The cannula may include a first bend configured to be located proximal of an aortic valve of the patient when the impeller assembly is pumping blood from the left ventricle to the aorta and a second bend configured to be located proximal of the first bend. The first bend and the second bend may be in a same plane.

### CROSS REFERENCE TO RELATED APPLICATIONS

This application claims the benefit of U.S. Provisional Patent Application Ser. No. 63/734,455 filed on Dec. 16, 2024, the disclosure of which is incorporated herein by reference.

### TECHNICAL FIELD

The present disclosure pertains to mechanical circulatory support devices and systems. More specifically, the present disclosure relates to devices, systems, and methods of and/or for positioning mechanical circulatory support devices and systems.

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, cardiac assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including ventricular assist devices.

A first example may include a mechanical circulatory support system having a motor, an impeller assembly in communication with the motor, the impeller assembly having an impeller housing and is configured to pump blood from a left ventricle of a heart of a patient to an aorta of the patient in response to actuation of the motor, a cannula extending distally from the impeller housing, and wherein the cannula may include a first bend configured to be located within or proximal of an aortic valve of the patient when the impeller assembly is pumping blood from the left ventricle to the aorta and a second bend configured to be located proximal of the first bend.

Alternatively or additionally to any of the examples above, the impeller housing may have a rigid structure and the cannula may have a flexible structure that is less stiff than the rigid structure.

Alternatively or additionally to any of the examples above, the cannula may be configured to extend through the aortic valve and into the left ventricle of the patient.

Alternatively or additionally to any of the examples above, the second bend may be configured to engage an aortic wall of the patient when the cannula extends into the left ventricle of the patient.

Alternatively or additionally to any of the examples above, the first bend may be configured to position the cannula within a central portion of a cross-section extending through the aortic valve when the impeller assembly is pumping blood from the left ventricle to the aorta.

Alternatively or additionally to any of the examples above, the first bend may define a first angle extending in a plane and the second bend may define a second angle extending in the plane.

Alternatively or additionally to any of the examples above, the first bend may define a first angle and the second bend may define a second angle equal to the first angle.

Alternatively or additionally to any of the examples above, the first bend may define a first angle in a range of 10 degrees to 50 degrees and the second bend defines a second angle in a range of 10 degrees to 50 degrees.

Alternatively or additionally to any of the examples above, a radius of curvature extending through the first bend and the second bend may be in a range of 10 millimeters (mm) to 40 mm.

Alternatively or additionally to any of the examples above, the cannula may be formed from a polymer material.

Alternatively or additionally to any of the examples above, the cannula may be formed from a shape set nickel-titanium alloy defining the first bend and the second bend.

Alternatively or additionally to any of the examples above, the impeller assembly may comprise an impeller and the impeller may rotate in response to actuation of the motor to draw blood from the left ventricle into the cannula and through the impeller housing into the aorta.

In a further example, a mechanical circulatory support system may include a blood pump configured to pump blood from a left ventricle of a heart of a patient to an aorta of the patient, a cannula extending distally from the blood pump, and wherein the cannula may include a first bend having a first angle in a plane and configured to direct the cannula through an aortic valve of the patient when the blood pump is pumping blood from the left ventricle to the aorta and a second bend having a second angle in the plane.

Alternatively or additionally to any of the examples above, the first bend and the second bend may be configured to be located proximal of the aortic valve when the blood pump is pumping blood from the left ventricle to the aorta.

Alternatively or additionally to any of the examples above, the first angle may be in a range of 10 degrees to 50 degrees and the second angle may be in a range of 10 degrees to 50 degrees.

Alternatively or additionally to any of the examples above, a radius of curvature extending through the first bend and the second bend may be in a range of 10 millimeters (mm) to 40 mm.

In a further example, a method may include inserting a mechanical circulatory support system into a vasculature of a patient, the mechanical circulatory support system comprising a blood pump and a cannula extending distally from the blood pump, the cannula having a first bend and a second bend located proximal of the first bend, delivering the mechanical circulatory support system through the vasculature to a heart of the patient, wherein the blood pump and the cannula are delivered through the vasculature within a sheath, and engaging the second bend with a wall of an aorta of the patient as the blood pump exits a distal end of the sheath.

Alternatively or additionally to any of the examples above, the method may further include delivering the cannula to a left ventricle of the heart of the patient while engaging the second bend with the wall of the aorta of the patient.

Alternatively or additionally to any of the examples above, the first bend may be in a plane and the second bend is in the plane.

Alternatively or additionally to any of the examples above, the blood pump may include an impeller assembly having an impeller housing and the second bend may be proximate a distal end of the impeller housing.

The above summary of some configurations is not intended to describe each disclosed configuration or every implementation of the present disclosure. The Figures, and Detailed Description, which follow, more particularly illustrate some of these configurations.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative circulatory support system;

FIG. 2 is a schematic partial cross-section view of anatomy and a schematic side view of an illustrative mechanical circulatory support (MCS) system within the anatomy;

FIG. 3 is a schematic cross-section view of a portion of an introducer sheath within a blood vessel and a portion of an illustrative MCS system in the introducer sheath and the blood vessel;

FIG. 4 is a schematic cross-section view of anatomy and a schematic side view of a portion of an illustrative MCS system within the anatomy;

FIG. 5 is a schematic side view of a portion of an illustrative MCS system;

FIG. 6 is a schematic side view of a portion of an illustrative MCS system;

FIG. 7 is a schematic side view of a portion of an illustrative MCS system;

FIG. 8 is a schematic side view of a portion of an illustrative MCS system;

FIG. 9 is a schematic side view of a portion of an illustrative MCS system;

FIG. 10 is a schematic side view of a portion of an illustrative MCS system;

FIG. 11 is a schematic side view of a portion of an illustrative MCS system; and

FIGS. 12-15 depict schematic views illustrating a portion of an illustrative technique for delivering an MCS system to a heart of a patient.

While the disclosure is amenable to various modifications and alternative forms, specifics thereof have been shown by way of example in the drawings and will be described in detail. The intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist or support devices are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support systems or devices may support a patient during percutaneous coronary interventions and/or other procedures.

Example cardiac circulatory support systems or devices include, but are not limited to, ventricular assist devices (VADs), total artificial hearts, intra-aortic balloon pumps (IABP), and extracorporeal membrane oxygenation (ECMO) devices. Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. A PVAD may be placed at a desired location of anatomy of a patient via percutaneous access and delivery, which may enable the PVAD to be used in emergency medicine, a cath lab, and/or other surgical and/or non-surgical settings.

In some examples, circulatory support systems may include a distal end having a flexible cannula that is configured to be inserted into a heart of a patient. A housing (e.g., a rigid housing) that may house one or more pumping components may extend in a proximal direction from a proximal end of the flexible cannula. A bend in the flexible cannula may direct the distal end of the flexible cannula through the aortic valve of the patient and into the left ventricle of the heart. As the flexible cannula and the housing extend out of a delivery or guide sheath proximate a wall of the aorta of the patient and the flexible cannula is inserted through the aortic valve, a proximal portion of the flexible cannula at a location proximate a distal end of the housing may engage the wall of the aorta and inadvertently bend. The portion of the flexible cannula proximate the distal end of the housing and that engages the wall of the aorta may be susceptible to kinking and/or weakening due to a transition in stiffness between the flexible cannula and the housing. As such, when circulatory support system is delivered to the heart of the patient multiple times, plastic deformation in the flexible cannula may occur at or near the connection between the flexible cannula and the housing.

The concepts disclosed herein facilitate delivering the circulatory support system to the heart of the patient and positioning the flexible cannula through the aortic valve of the patient and into the left ventricle, while mitigating damage to the cannula and forces between the cannula and the wall of the aorta of the patient. In one example, the cannula may include a bend proximate to the proximal end of the cannula that facilitates delivering the cannula to the heart of the patient and reduces forces between the cannula and the aortic wall of the patient. In some examples, the bend may be a second bend positioned between a first bend configured to direct the cannula through the aortic valve and into the left vertical and a proximal end of the cannula. Other suitable configurations of the cannula are contemplated.

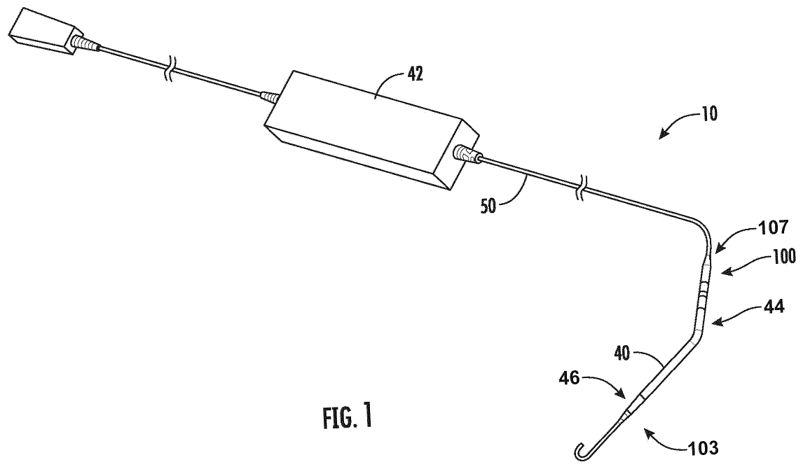

FIG. 1 depicts a schematic view of an illustrative circulatory support system 10 (e.g., a mechanical circulatory support (MCS) system). The system 10 may include a percutaneous support device (e.g., a PVAD, such as a blood pump 100 having a proximal end 107 and a distal end 103), a cannula 40 having a proximal end 44 and a distal end 46, an elongate tube 50, and a delivery or guide sheath (not shown). In some examples, the system 10 may include a guidewire, but this is not required. The delivery or guide sheath, when included, may facilitate percutaneous delivery of the blood pump 100 and the cannula 40 to a target location within a patient (e.g., a target location within a heart of a patient). When positioned at a target site in a heart of a patient, the blood pump 100 may be configured to pump blood from a ventricle of the heart to vasculature of the patient.

The cannula 40 may be a component of the blood pump 100 and/or a separate component coupled with the blood pump 100. In some examples, although the cannula 40 may be discussed as extending from the blood pump 100 with the proximal end 44 of the cannula 40 at or proximate the distal end 103 of the blood pump 100, the cannula 40 may be part of the blood pump 100 (e.g., formed from a component of the blood pump 100, such as an impeller housing and/or other suitable component) with the distal end 46 of the cannula 40 at the distal end 103 of the blood pump 100, but other suitable configurations are contemplated.

The system 10 may further include a proximal housing 42 coupled with (e.g., connected to) the elongate tube 50 (e.g., a catheter and/or other suitable elongate tube), where the elongate tube 50 may be coupled with the blood pump 100 (e.g., a distal end of the elongate tube 50 may be coupled with the proximal end 107 of the blood pump 100) and one or more wires and/or shafts coupled with the blood pump 100 may extend proximally from the blood pump 100 through the tube 50. In some examples, at least the proximal housing 42, the elongate tube 50, and/or other components of the system 10 may be configured to facilitate delivering the blood pump 100 and the cannula 40 to the target location or site within the patient. The elongate tube 50 and the proximal housing 42 may be configured to be positioned at least partially exterior of the patient when the blood pump 100 and the cannula 40 are at the target location. In some examples, the guidewire, when included, may also be used to facilitate the delivery of the blood pump 100 and/or the cannula 40.

Although not shown, the system 10 may additionally or alternatively include one or both of a starter tube and a starter tube flushing line. The starter tube, when included, may couple with a cannula delivery tool (e.g., a delivery sheath) which may be configured to receive the cannula 40 prior to and/or during delivery of the blood pump 100 and the cannula 40 to a target site.

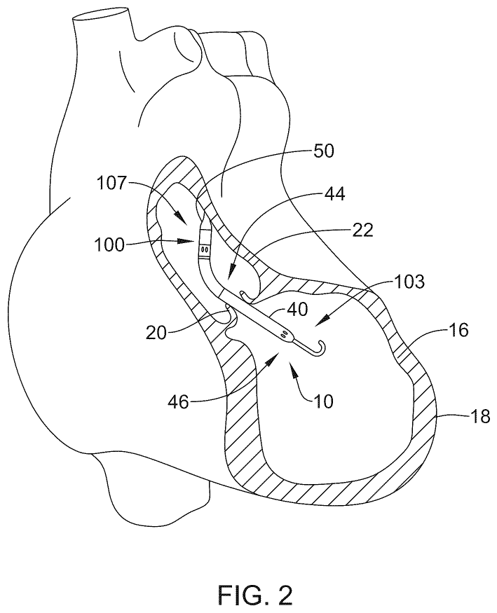

FIG. 2 depicts a schematic view of an illustrative positioning of the blood pump 100 (e.g., a percutaneous circulatory support device, such as a PVAD, etc.) in anatomy of a patient (e.g., in a left ventricle 16 in a heart 18). As depicted in FIG. 2, the blood pump 100 may be positioned with the distal end 103 thereof (e.g., when including or combined with the cannula 40) located in the left ventricle 16 of the heart 18 and the proximal end 107 of the blood pump 100 in an aorta 22, such that the blood pump 100 and the cannula 40 in combination extends across an aortic valve 20 between the left ventricle 16 and the aorta 22. With the blood pump 100 extending from the left ventricle 16 to the aorta 22 of the patient, the blood pump 100 may be configured to pump blood from the left ventricle 16 into the aorta 22 (e.g., into the ascending aorta) to assist or support blood flow circulation. Other suitable positions of the blood pump 100 relative to the anatomy of the patient are contemplated and include, but are not limited to, the distal end 103 of the blood pump 100 being positioned in or in fluid communication with a right ventricle of the heart 18 with the proximal end 107 of the blood pump 100 being positioned in a pulmonary artery.

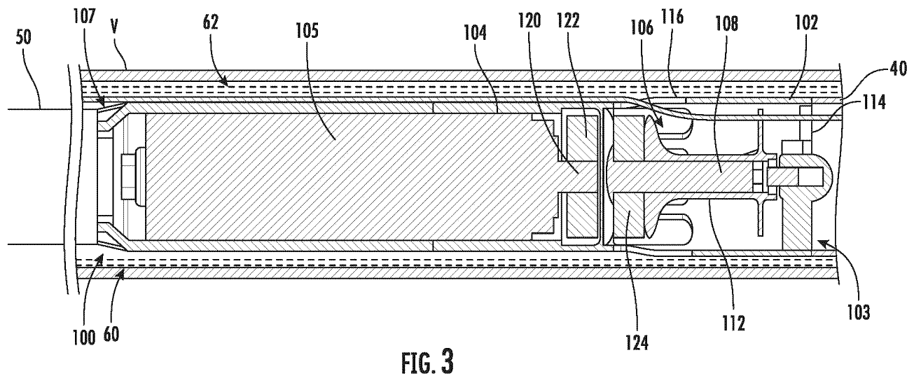

FIG. 3 depicts a schematic cross-section view of a body portion 62 of a delivery or guide sheath 60 (hereinafter “guide sheath 60”) extending into a blood vessel V with the blood pump 100 inserted into the guide sheath 60. The blood pump 100 may include and/or may be coupled with the elongate tube 50 extending proximally from a proximal end 107 of the blood pump 100 to a location outside of the blood vessel V and the guide sheath 60. When positioning the blood pump 100 within the patient, the blood pump 100 may be advanced through the blood vessel V and positioned in or at a target location, such as a target cardiac location (e.g., in the aorta 22, across the aortic valve 20, and/or into the left ventricle 16), via the guide sheath 60. While the guide sheath 60 is illustrated in FIG. 3 with the use of the blood pump 100, various other medical devices may be used in conjunction with the guide sheath 60.

The blood pump 100 may include one or more components. In some examples, the blood pump 100 may include an impeller housing 102 and a motor housing 104. The impeller housing 102 and the motor housing 104 may be integrally or monolithically constructed. Alternatively, the impeller housing 102 and the motor housing 104 may be separate components. The impeller housing 102 may carry an impeller assembly 106 therein. The impeller assembly 106 may include an impeller shaft 108 and an impeller 112 that rotates relative to the impeller housing 102 to drive or pump blood from the left ventricle 16 of the heart 18 of the patient (e.g., via the cannula 40 of or in fluid communication with the blood pump 100) to the aorta 22 or portion of the anatomy in communication with the aorta (e.g., pump blood through the blood pump 100 to an ascending aorta). In some examples, the impeller shaft 108 and the impeller 112 may be integrally formed, whereas, in other examples the impeller shaft 108 and the impeller 112 may be separate components.

Rotation of the impeller 112 may cause blood to flow from a blood inlet 114 formed on the impeller housing 102 (e.g., where the blood may enter the blood inlet 114 directly from the left ventricle and/or via the cannula 40), through the impeller housing 102, and out of a blood outlet 116 formed on the impeller housing 102. As shown in FIG. 3, the blood inlet 114 may be formed on an end portion of the impeller housing 102 and the blood outlet 116 may be formed on a side portion of the impeller housing 102. In other examples, the blood inlet 114 and/or the blood outlet 116 may be formed on or at other portions of the impeller housing 102 or other components of the system 10.

The impeller housing 102 of the impeller assembly 106 may be coupled to the cannula 40 and the cannula 40 may extend distally from the impeller housing 102 (e.g., extending distally from the distal end 103 of the blood pump 100), and the cannula 40 may receive blood from the left ventricle and deliver the blood to the blood inlet 114. Alternatively or additionally, the impeller housing 102 may be further elongated than depicted in FIG. 3 and define all of or at least part of the cannula 40 extending into the left ventricle of the patient. Other suitable configurations of the impeller housing 102 and/or the cannula 40 are contemplated.

The motor housing 104 may carry a motor 105, and the motor 105 may be configured to rotatably drive the impeller 112 relative to the impeller housing 102 when the motor 105 is actuated. In some examples, the motor 105 may rotate a drive shaft 120 coupled with a driving magnet 122. Rotation of the driving magnet 122 may cause rotation of a driven magnet 124 that may be coupled with and/or may be part of the impeller assembly 106. More specifically, in configurations incorporating the impeller shaft 108, the impeller shaft 108 and the impeller 112 may be configured to rotate with the driven magnet 124. In other configurations, the motor 105 and/or the drive shaft 120 may be coupled to the impeller assembly 106 directly and/or via other suitable components.

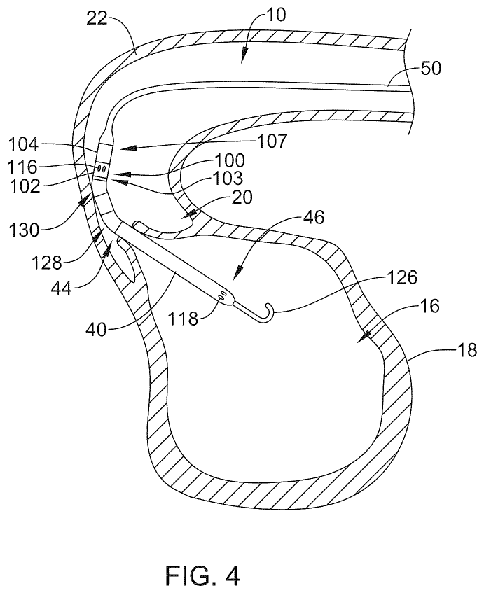

FIG. 4 depicts a schematic cross-section view of anatomy of a patient with a schematic side view of a portion of an illustrative configuration of the system 10 extending within the anatomy, where the cannula 40 includes a first bend 128 and a second bend 130. The anatomy schematically depicted in FIG. 4 includes, among other anatomy, the aorta 22, the aortic valve 20, the heart 18, and the left ventricle 16.

When the system 10 is positioned within the anatomy of the patient, for example as depicted in FIG. 4, the blood pump 100 may be positioned at the aorta 22, the cannula 40 may extend distally from the distal end 103 of the blood pump 100, across the aortic valve 20, and into the left ventricle 16 of the heart 18 (e.g., schematically depicted with chambers other than the left ventricle 16 omitted), and the elongate tube 50 may extend proximally from a proximal end 107 of the blood pump 100 through the aorta 22 and eventually out of patient (e.g., via the femoral artery and/or other suitable vessel access site). When so positioned, inflow openings 118 of the cannula 40 may be positioned within the left ventricle 16 and the blood outlet 116 may be located in the aorta 22.

An atraumatic distal tip 126 may form a distal end 46 of the cannula 40 and/or may extend distally from the distal end 46 of the cannula 40, but the atraumatic distal tip 126 may be omitted. When included, the atraumatic distal tip 126 may be bent to form a rounded distal end, but other suitable configurations are contemplated.

The blood pump 100 and/or the cannula 40 may be configured to facilitate delivering and positioning the blood pump 100 and/or the cannula 40 in a desired manner within the heart 18 and aorta 22 of the patient. For example, the blood pump 100 and/or the cannula 40 may be configured to deliver the cannula 40 and/or the blood pump 100 into the aorta 22 from the guide sheath 60 (not depicted in FIG. 4), center the cannula 40 within the aortic valve 20, position the blood outlet 116 in the aorta 22, and position the inflow openings 118 within the left ventricle 16.

To facilitate delivering and/or positioning the blood pump 100 and/or the cannula 40 within the aorta 22 and the heart 18 or other suitable target location in a desired manner, the blood pump 100 and/or cannula 40 may include one or more bends. In some examples, the one or more bends may be pre-formed bends in the blood pump 100 and/or cannula 40, which may form an intended angle when no exterior forces are acting on the blood pump 100 and/or the cannula 40. In some examples, the one or more bends may be heat set into material forming a housing of the blood pump 100 and/or material forming the cannula 40. Other suitable techniques for preforming or facilitating the one or more bends in the blood pump 100 and/or the cannula 40 are contemplated.

As depicted in FIG. 4, the cannula 40 may include the first bend 128. In some examples, the first bend 128 may be configured to facilitate crossing the aortic valve 20 with the cannula 40 and mitigate damage to the aortic valve 20 as the cannula 40 is positioned within the left ventricle 16. In some examples, the first bend 128 may be configured to position the cannula 40 or other suitable component of the system 10 in a central portion of a cross-section of an opening through the aortic valve 20 (e.g., where the opening may be entirely or at least partially defined by leaflets of the aortic valve 20 when the leaflets are in an opened position), where the cross-section may be taken at an axial location of the opening at which leaflets of the aortic valve 20 coapt with or are intended to coapt one another when in a closed position. Further, the first bend 128 and the second bend 130 of the cannula 40 may be configured to be located proximal of the aortic valve 20 when the blood pump 100 and/or the cannula 40 are at a target location within the heart 18 and the blood pump 100 is pumping blood from the left ventricle 16 to the aorta 22.

When delivering the cannula 40 across the aortic valve 20 and into the left ventricle 16, a proximal end 44 of the cannula 40 proximate the distal end 103 of the blood pump 100 may engage a wall of the aorta 22 as the cannula 40 and the blood pump 100 exit the guide sheath 60 and the cannula 40 crosses the aortic valve 20 and enters the left ventricle 16. When the cannula 40 is formed to be straight between the first bend 128 and the distal end 103 of the blood pump 100 (e.g., a distal end of the impeller housing 102 or other suitable housing of the blood pump 100), relatively large forces may be required to deliver the cannula 40 and the blood pump 100 out of the guide sheath 60 due, at least in part, to a positioning of the guide sheath 60 at or near the wall of the aorta 22, an angle the cannula 40 and the blood pump 100 may need to traverse when exiting the guide sheath 60, and a stiffness or rigidity of the blood pump 100. In some examples, the force between the cannula 40 and the wall of the aorta 22 may cause the cannula 40 to bend and kink at or proximate where the cannula 40 engages the blood pump 100 due at least in part to a change in stiffness between the cannula 40 and the housing of the blood pump 100.

Bending and/or kinking of the cannula 40 may weaken the material of the cannula 40 and when the blood pump 100 has to be repeatedly delivered to the patient, the weakened material of the cannula 40 proximate the blood pump 100 may cause plastic deformation of the cannula 40 resulting in damage to the cannula 40 (e.g., cracking of the cannula 40, etc.) Further, when the cannula 40 must be bent at a location and/or angle at which it is not intended to be bent for delivering the cannula 40 and/or the blood pump 100 from the guide sheath 60 into the aorta 22, the forces required to deliver the cannula 40 and/or the blood pump 100 out of the guide sheath 60 and into the aorta 22 may cause trauma to the aorta 22 and/or other anatomy of the patient.

To facilitate delivery of the cannula 40 and/or the blood pump 100 into the aorta 22 of the patient, mitigate damage to the cannula 40, mitigate forces on the aorta 22 or other anatomy of the patient, and/or for other purposes, the cannula 40 may include the second bend 130 between the first bend 128 and the distal end 103 of the blood pump 100 (e.g., a distal end of the impeller housing 102). When the cannula 40 includes the second bend 130, the second bend 130 may be configured to direct the cannula 40 away from the aorta 22 as the cannula 40 is delivered into the aorta 22 from the guide sheath 60 and to engage the aorta 22 as the blood pump 100 exits the guide sheath 60.

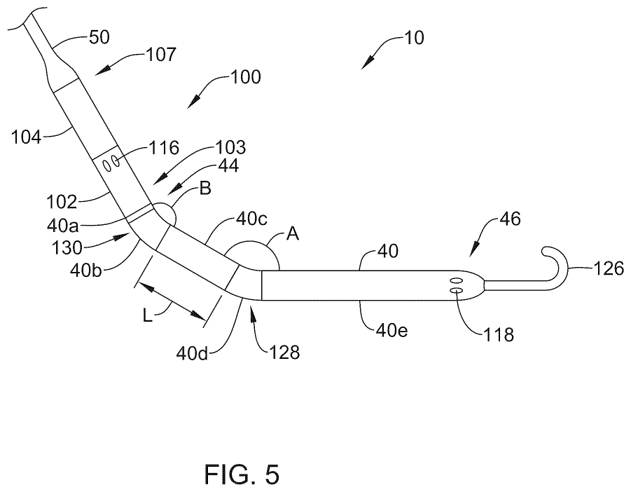

FIG. 5 depicts a schematic side view of a portion of an illustrative configuration of the system 10 with the cannula 40 having a first bend 128 and a second bend 130. Although the first bend 128 and the second bend 130 are depicted in FIG. 5 and described as being formed in the cannula 40, the first bend 128 and the second bend 130 may be formed in other suitable components of the system 10 including, but not limited to, the blood pump 100, the impeller housing 102, the motor housing 104, and/or other suitable components. The first bend 128 and/or the second bend 130 may be configured to prevent or mitigate unintended bending or kinking in the cannula 40 at or proximate to the housing of the blood pump 100 that may be caused by an abrupt change in stiffness between the cannula 40, which may be flexible, and the housing of the blood pump 100, which may be more stiff or rigid than the cannula 40.

The housing of the blood pump (e.g., the motor housing 104 and/or the impeller housing 102) may be formed from any suitable material. For example, the impeller housing 102 and/or the motor housing 104 may be formed from a rigid material, rigid metal material, a rigid ceramic, a rigid polymer, a stainless steel, and/or other suitable materials. In one example, the impeller housing 102 may be formed from a rigid structure having a first stiffness.

The cannula 40 (e.g., an entirety of the cannula 40, the cannula 40 at the first bend 128, the cannula 40 at the second bend 130, and/or at other locations along the cannula 40 and/or the blood pump 100) may be formed from any suitable material configured to facilitate delivering the system 10 to the target location in the heart 18 of the patient and forming the first bend 128 and/or second bend 130 when the blood pump 100 and the cannula 40 are positioned at the target location within the aorta 22 and/or the heart 18. The material of the cannula 40 may be selected based on one or more considerations including, but not limited to, kink resistance, lubricity, flexibility, rigidity or stiffness, resilience, shape memory properties, and/or other suitable considerations. In one example, the cannula 40 may be formed from a flexible structure having a second stiffness that is less stiff than the first stiffness of the rigid structure forming the impeller housing 102.

Example suitable materials for the cannula 40 may include, but are not limited to, metals, polymers, shape memory materials, shape set materials, nickel-titanium alloys (e.g., NITINOL and/or other suitable nickel-titanium alloys), stainless steel, polytetrafluoroethylene (PTFE), polyamid (e.g., VESTAMID and/or other suitable polyamid material) and/or other suitable materials. In some examples, the cannula 40 and/or portions thereof may be formed from a shape set nickel-titanium alloy defining the first bend 128 and the second bend 130. In some examples, the cannula 40 and/or portions thereof may be formed from stainless steel. In some examples, the cannula 40 and/or portions thereof may be formed from a polymer material. In some examples, the cannula 40 and/or portions thereof may be formed from a metal inner tube and polymer outer tube.

The material may be formed in any suitable manner that is configured to form the illustrative configurations of the cannula 40 discussed herein. For example, the material of the cannula 40 may be formed in a tubular manner, formed into a braid, formed into a coil, formed into a hypotube, formed into a slotted hypotube, reflowed, molded, coated, and/or formed in one or more other suitable manners. In some examples, the cannula 40 may be formed from a shape set nickel-titanium alloy hypotube having laser cuts. In some examples, the cannula 40 may be formed from a shape set nickel-titanium alloy hypotube having a coil configuration. In some examples, the cannula 40 may be formed from a shape set polymer. In some examples, the cannula 40 may be formed from a metallic or polymer inner tube having a braid, coil, and/or slotted hypotube configuration and one or more both of a reflowed, molded, or coated polymer outer tube or layer and a reflowed, molded, or coated inner tube or layer. In some examples, the cannula 40 or at least portions at or proximate the first bend 128 and/or the second bend 130 may be formed from a shape memory nickel-titanium alloy hypotube that is heat set to transition from a delivery configuration (e.g., a straight or non-bent configuration) to an operational configuration (e.g., a configuration with the first bend 128 and the second bend 130, etc.) at a transition temperature. When so configured, the transition temperature may be a temperature in a range from room temperature to a body temperature, a temperature in a range from 20 degrees Celsius to 40 degrees Celsius. In one example, the transition temperature may be 37 degrees Celsius, but other suitable transition temperatures are contemplated.

The cannula 40 may have one or more segments or portions defining features of the cannula 40. In one example, when the cannula 40 is coupled with the distal end 103 of the blood pump 100 (e.g., coupled with a distal end of the impeller housing 102), a first segment 40a at a proximal end 44 of the cannula 40 may couple with the distal end 103 of the blood pump 100, a second segment 40b of the cannula 40 may extend distally from the first segment 40a and define the second bend 130, a third segment 40c of the cannula 40 may extend distally from the second segment 40b, a fourth segment 40d may extend distally from the third segment 40c and define the first bend 128, and a fifth segment 40e may extend distally from the fourth segment 40d. In some examples, the fifth segment 40e of the cannula 40 may include or form the inflow openings 118 or the inflow openings 118 may be separate from the fifth segment 40e (e.g., the inflow openings 118 may be part of a sixth portion of the cannula 40 and/or may be separate from the cannula 40). In some examples, all of or at least one or more of the first segment 40a, the third segment 40c, and the fifth segment 40e may be configured to be straight, without defining a curve or bend in the longitudinal direction. Other suitable configurations of the cannula 40 with additional and/or alternative segments or portions are contemplated.

The segments or portions of the cannula 40 may have any suitable axial lengths. In some examples, the first segment 40a of the cannula 40 may have a length in a range of 0 millimeters (mm) (0 inches) to 5 mm (0.197 inches), the second segment 40b of the cannula 40 may have a length in a range of 1 mm (0.039 inches) to 30 mm (1.181 inches), the third segment 40c of the cannula 40 may have a length in a range of 0 mm (0 inches) to 40 mm (1.575 inches), the fourth segment 40d of the cannula 40 may have a length in a range of 1 mm (0.039 inches) to 30 mm (1.181 inches), and the fifth segment 40e of the cannula 40 may have a length in a range of 0 mm (0 inches) to 50 mm (1.969 inches). The first segment 40a, the third segment 40c, and the fifth segment 40e may have lengths of 0 mm (0 inches) when such portions are omitted and the curves defined by the second segment 40b and the fourth segment 40d run together without an extension portion and/or an intervening portion. Other suitable lengths of the segments of the cannula 40 are contemplated.

The second bend 130 may be positioned at or proximate to the housing of the blood pump 100 to prevent over-bending or kinking of the cannula 40 proximate the housing of the blood pump 100. For example, due to a change in stiffness between the cannula 40 and the housing of the blood pump 100 (e.g., the impeller housing 102) positioning the second bend 130 at or proximate the housing of the blood pump 100 may reduce bending forces on the cannula 40 as the blood pump 100 exits the guide sheath 60 and the cannula 40 engages the wall of the aorta 22.

The first bend 128 and the second bend 130 may define angles in a same plane. For example, a single plane may extend through at least a central axis of the second segment 40b, the third segment 40c, and the fourth segment 40d of the cannula 40. In some examples, a plane may extend through a central axis of the housing of the blood pump 100, the first segment 40a, the second segment 40b, the third segment 40c, the fourth segment 40d, and the fifth segment 40e. Other suitable configurations of the blood pump 100 and/or the cannula 40 are contemplated.

The first bend 128 and the second bend 130 may have any suitable configurations. In some examples, the first bend 128 and/or the second bend 130 may be configured individually and/or relative to one another such that the bends 128, 130 are configured to facilitate delivering the blood pump 100 and/or the cannula 40 to a target location and centering the cannula 40 in the aortic valve 20. In some examples, an angle A of the first bend 128, an angle B of the second bend 130, and a length L of the third segment 40c may be configured to form a desired radius of curvature through the first bend 128 and the second bend 130. In some examples, the third segment 40c may be omitted and a single bend having the desired radius of curvature may be utilized.

The one or more bends of the cannula 40 and/or the blood pump 100 may be configured to form any suitable radius of curvature in the cannula 40 and/or the blood pump 100. For example, a centerline radius of curvature extending through either or both of the first bend 128 and the second bend 130 may be in a range of 2 mm (0.079 inches) to 30 mm (1.181 inches, and/or other suitable ranges. In some examples, the centerline length through the first bend, the straight section, and the second bend combined may be in a range of 10 mm (0.394 inches) to 40 mm (1.575 inches) and/or other suitable ranges.

The angle A of the first bend 128 and the angle B of the second bend 130 may be combined to form an angle extending between the first segment 40a of the cannula 40 or the housing of the blood pump 100 and the fifth segment 40e of the cannula and having any suitable value. For example, the combined angle of the first bend 128 and the second bend 130 may be a value in a range of 5 degrees and 90 degrees and/or a value in one or more other ranges. In one example, the value of the combined angle of the first bend 128 and the second bend 130 may be 60 degrees. The combined angle of the first bend 128 and the second bend 130, however, may have other suitable values.

The angle A of the first bend 128 extending between the third segment 40c of the cannula 40 and the fifth segment 40e may have any suitable value. For example, the angle A of the first bend 128 may be a value in a range of 5 degrees to 90 degrees, in a range of 10 degrees to 50 degrees, and/or in one or more other suitable ranges. The angle B of the second bend 130 extending between the first segment 40a of the cannula 40 or the housing of the blood pump 100 and the third segment 40c may have any suitable value. For example, the angle B of the second bend 130 may be a value in a range of 5 degrees to 90 degrees, 10 degrees to 50 degrees, and/or other suitable range. The angle A of the first bend 128 and the angle B of the second bend 130 may have a same value, the angle A of the first bend 128 may be greater than the angle B of the second bend 130, or the angle A of the first bend 128 may be less than the angle B of the second bend 130. Other suitable values for the angles of the first bend 128 and the second bend 130 are contemplated.

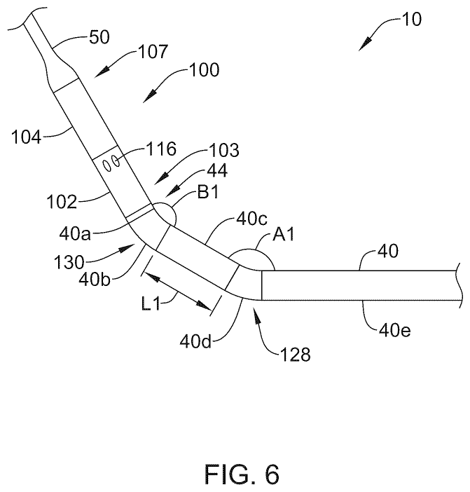

FIGS. 6-11 schematically depict different illustrative configurations of the segments 40a-40e of the cannula 40 forming different values of the angle A of the first bend 128, the angle B of the second bend 130, and the length L of the third segment 40c of the cannula 40. The combined configurations of the first bend 128, the second bend 130, and the third segment 40c may be designed to achieve a desired combined angle in the cannula 40 and/or a desired radius of curvature through the first bend 128 and the second bend 130 (e.g., through a central axis of the cannula 40 at the first bend 128 and the second bend 130). Although example values of the first angle A of the first bend 128, the second angle B of the second bend 130, the length L of the third segment 40c of the cannula 40, a combined angle of the cannula 40, and a radius of curvature through the first bend 128 and the second bend 130 are discussed with respect to FIGS. 6-11, other suitable values are contemplated.

FIG. 6 schematically depicts the cannula 40 coupled with and extending in a distal direction from the housing of the blood pump 100 (e.g., from the impeller housing 102). The first bend 128 at the fourth segment 40d of the cannula 40 may have an angle A1, the second bend 130 at the second segment 40b of the cannula 40 may have an angle B1, and the third segment 40c of the cannula 40 may have a length L1. In the example configuration depicted in FIG. 6, the angle A1 may be 30 degrees, the angle A2 may be 30 degrees, the length L1 may be 12.83 mm (0.505 inches), which may result in a combined angle of 60 degrees in the cannula 40 and may form a radius of curvature extending through the first bend 128 and the second bend 130 of 11.2 mm (e.g., 0.441 inches).

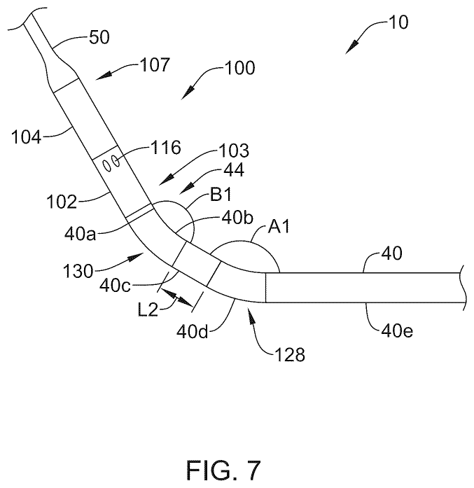

FIG. 7 schematically depicts the cannula 40 coupled with and extending in a distal direction from the housing of the blood pump 100 (e.g., from the impeller housing 102). The first bend 128 at the fourth segment 40d of the cannula 40 may have the angle A1, the second bend 130 at the second segment 40b of the cannula 40 may have the angle B1, and the third segment 40c of the cannula 40 may have a length L2. In the example configuration depicted in FIG. 7, the angle A1 may be 30 degrees, the angle A2 may be 30 degrees, the length L2 may be 4.85 mm (0.191 inches), which may result in a combined angle of 60 degrees in the cannula 40 and form a radius of curvature extending through the first bend 128 and the second bend 130 of 18.8 mm (e.g., 0.741 inches).

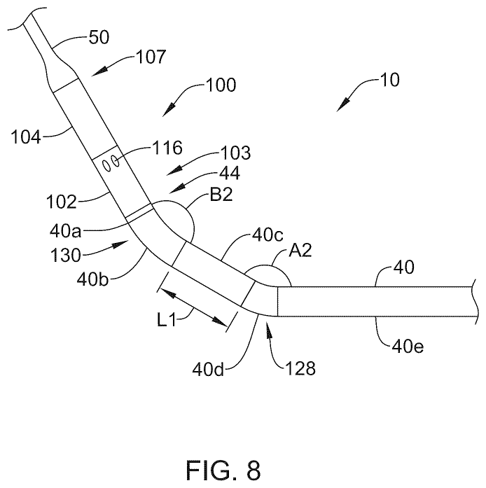

FIG. 8 schematically depicts the cannula 40 coupled with and extending in a distal direction from the housing of the blood pump 100 (e.g., from the impeller housing 102). The first bend 128 at the fourth segment 40d of the cannula 40 may have an angle A2, the second bend 130 at the second segment 40b of the cannula 40 may have an angle B2, and the third segment 40c of the cannula 40 may have the length L1. In the example configuration depicted in FIG. 8, the angle A2 may be 15 degrees, the angle B2 may be 45 degrees, the length L1 may be 12.83 mm (0.505 inches), which may result in a combined angle of 60 degrees in the cannula 40 and form a radius of curvature extending through the first bend 128 and the second bend 130 of 11.2 mm (e.g., 0.441 inches).

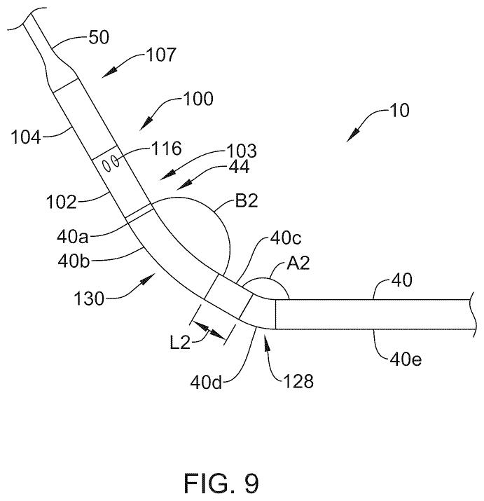

FIG. 9 schematically depicts the cannula 40 coupled with and extending in a distal direction from the housing of the blood pump 100 (e.g., from the impeller housing 102). The first bend 128 at the fourth segment 40d of the cannula 40 may have the angle A2, the second bend 130 at the second segment 40b of the cannula 40 may have the angle B2, and the third segment 40c of the cannula 40 may have a length L2. In the example configuration depicted in FIG. 9, the angle A2 may be 15 degrees, the angle B2 may be 45 degrees, the length L2 may be 4.85 mm (0.191 inches), which may result in a combined angle of 60 degrees in the cannula 40 and form a radius of curvature extending through the first bend 128 and the second bend 130 of 18.8 mm (e.g., 0.741 inches).

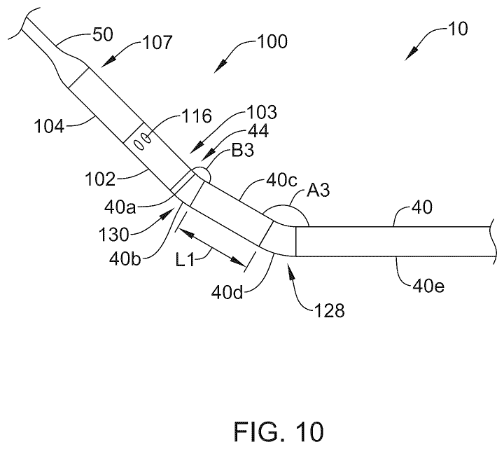

FIG. 10 schematically depicts the cannula 40 coupled with and extending in a distal direction from the housing of the blood pump 100 (e.g., from the impeller housing 102). The first bend 128 at the fourth segment 40d of the cannula 40 may have an angle A3, the second bend 130 at the second segment 40b of the cannula 40 may have an angle B3, and the third segment 40c of the cannula 40 may have the length L1. In the example configuration depicted in FIG. 10, the angle A3 may be 15 degrees, the angle B3 may be 45 degrees, the length L1 may be 12.83 mm (0.505 inches), which may result in a combined angle of 60 degrees in the cannula 40 and form a radius of curvature extending through the first bend 128 and the second bend 130 of 11.2 mm (e.g., 0.441 inches).

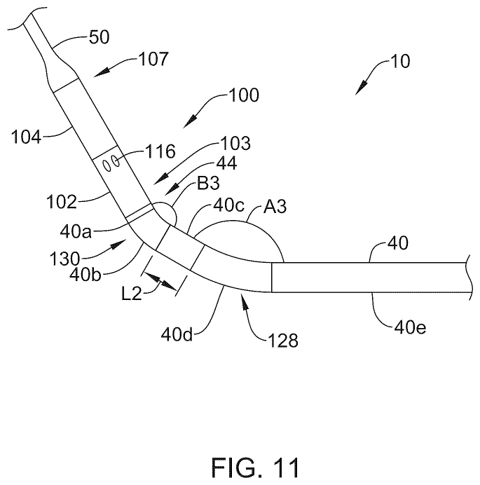

FIG. 11 schematically depicts the cannula 40 coupled with and extending in a distal direction from the housing of the blood pump 100 (e.g., from the impeller housing 102). The first bend 128 at the fourth segment 40d of the cannula 40 may have the angle A3, the second bend 130 at the second segment 40b of the cannula 40 may have the angle B3, and the third segment 40c of the cannula 40 may have a length L2. In the example configuration depicted in FIG. 11, the angle A3 may be 15 degrees, the angle B3 may be 45 degrees, the length L2 may be 4.85 mm (0.191 inches), which may result in a combined angle of 60 degrees in the cannula 40 and form a radius of curvature extending through the first bend 128 and the second bend 130 of 18.8 mm (e.g., 0.741 inches).

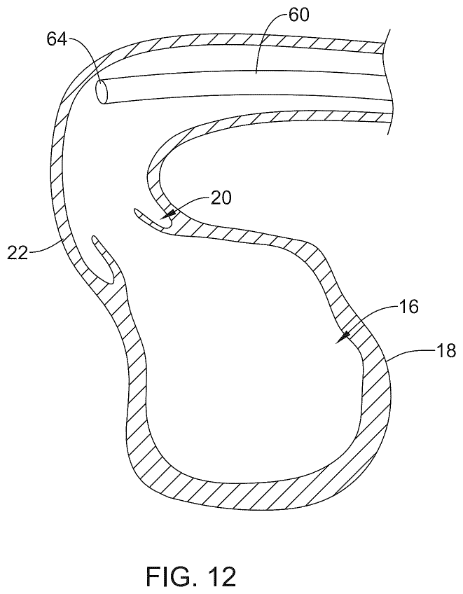

FIGS. 12-15 schematically depict an illustrative configuration of a method or technique for inserting and/or delivering the cannula 40 and the blood pump 100 of the system 10 to anatomy of a patient (e.g., into and/or through vasculature of the patient and the heart 18 of the patient). The cannula 40 of the system 10 may include the first bend 128 and the second bend 130 in the cannula 40, where the second bend 130 may be proximate a distal end of the impeller housing 102. As depicted in FIGS. 12-15, the method may include delivering the system 10 to the anatomy of the patient through the guide sheath 60.

FIG. 12 schematically depicts inserting the guide sheath 60 into the aorta 22 of the patient. In some examples, a distal end 64 of the guide sheath 60 may include an opening positioned proximate a wall of the aorta 22 and through which the cannula 40 and/or the blood pump 100 are delivered into the aorta 22. The guide sheath 60 may be positioned in the vasculature at the aorta 22 prior to inserting the cannula 40 through the guide sheath 60 or the blood pump 100 of the system 10 and/or the cannula 40 may be advanced through the vasculature into the aorta 22 with the guide sheath 60.

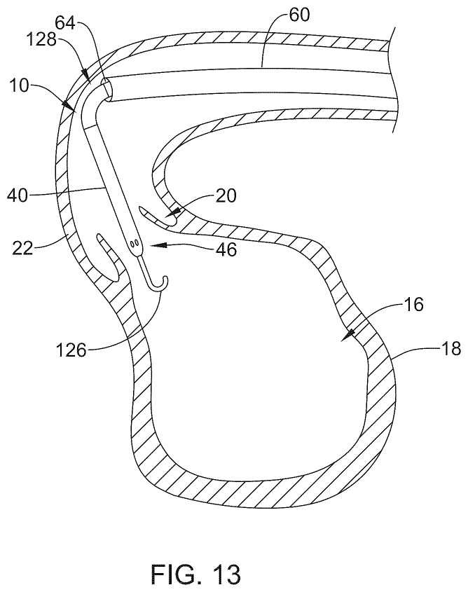

FIG. 13 schematically depicts delivering the cannula 40 of the system 10 through distal end 64 of the guide sheath 60 and into the aorta 22 of the vasculature of the patient. As depicted in FIG, 13, the first bend 128 of the cannula 40 may exit the guide sheath 60 proximate the wall of the aorta 22 to facilitate directing the cannula 40 to the aortic valve 20 and into the left ventricle 16 of the heart 18.

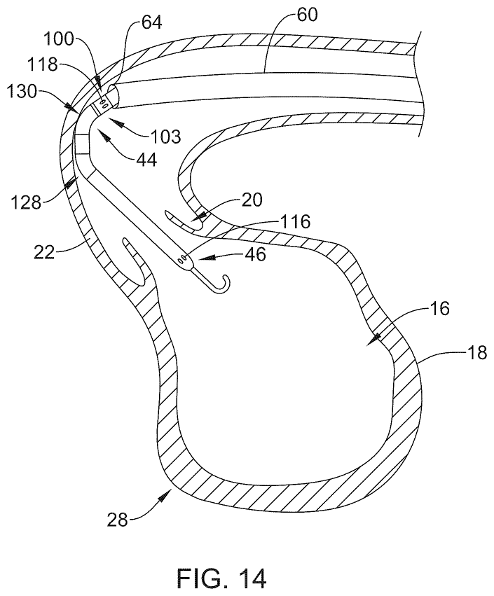

FIG. 14 schematically depicts delivering the cannula 40 and the blood pump 100 of the system 10 through distal end 64 of the guide sheath 60 and into the aorta 22 of the vasculature of the patient. As depicted in FIG. 14, the first bend 128 of the cannula 40 may direct the cannula 40 though the aortic valve 20 an into the left ventricle of the heart 18. The second bend 130 of the cannula 40 may exit the guide sheath 60 proximate the wall of the aorta 22, engage the wall of the aorta 22, and facilitate delivering or inserting the blood pump 100 into the aorta 22 and out of the distal end 64 of the guide sheath 60. As discussed herein, the first bend 128 and the second bend 130 may be in a same plane. In some example configurations, the second bend 130 of the cannula 40 may engage the wall of the aorta 22 as the blood pump 100 is exiting the distal end 64 of the guide sheath 60 and direct the cannula 40 and blood pump 100 toward the aortic valve 22 as the blood pump 100 advances in a distal direction relative to the guide sheath 60. In some example configurations, the second bend 130 of the cannula 40 may engage the wall of the aorta 22 as the cannula is delivered into the left ventricle 16 of the heart 18 of the patient.

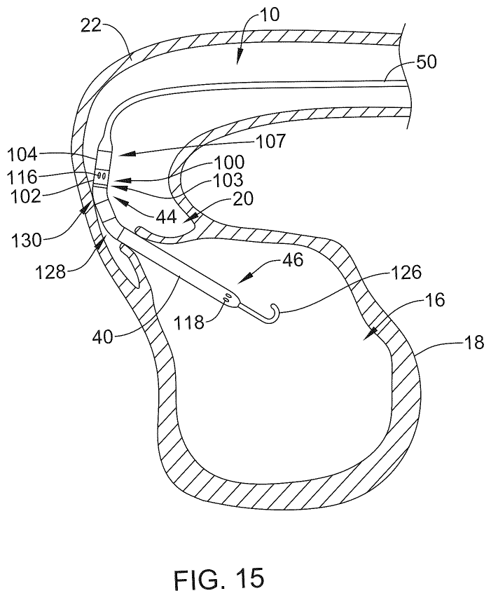

FIG. 15 schematically depicts the cannula 40 delivered to a target location in the left ventricle 16 of the heart 18 of the patient and the blood pump 100 delivered to a target location in the aorta 22 of the patient. In some examples, once the cannula 40 and the blood pump 100 have been delivered to the respective target locations, the guide sheath 60 may be removed from the vasculature of the patient. Alternatively, the guide sheath 60 may remain in the vasculature of the patient and/or may be advanced proximally (e.g., withdrawn) at one or more other suitable instances. Additional and/or alternative steps to those described with respect to FIGS. 12-15 may be utilized to deliver the cannula 40 and/or the blood pump 100 to the target location within anatomy of the patient.

The materials that can be used for the various components of the devices and the various elements thereof disclosed herein may include those commonly associated with medical devices. In some instances, the devices described herein, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyetherester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility may be imparted into the devices and/or other elements disclosed herein. For example, the devices described herein, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The scope of the disclosure is, of course, defined in the language in which the appended claims are expressed.

What is claimed is:

1. A mechanical circulatory support system comprising:
a motor;
an impeller assembly in communication with the motor, the impeller assembly having an impeller housing and is configured to pump blood from a left ventricle of a heart of a patient to an aorta of the patient in response to actuation of the motor;
a cannula extending distally from the impeller housing, and
wherein the cannula comprises:
a first bend configured to be located within or proximal of an aortic valve of the patient when the impeller assembly is pumping blood from the left ventricle to the aorta, and
a second bend configured to be located proximal of the first bend.

2. The system claim 1, wherein the impeller housing has a rigid structure and the cannula has a flexible structure that is less stiff than the rigid structure.

3. The system of claim 1, wherein the cannula is configured to extend through the aortic valve and into the left ventricle of the patient.

4. The system of claim 3, wherein the second bend is configured to engage an aortic wall of the patient when the cannula extends into the left ventricle of the patient.

5. The system of claim 1, wherein the first bend is configured to position the cannula within a central portion of a cross-section extending through the aortic valve when the impeller assembly is pumping blood from the left ventricle to the aorta.

6. The system of claim 1, wherein the first bend defines a first angle extending in a plane and the second bend defines a second angle extending in the plane.

7. The system of claim 1, wherein the first bend defines a first angle and the second bend defines a second angle equal to the first angle.

8. The system of claim 1, wherein the first bend defines a first angle in a range of 10 degrees to 50 degrees and the second bend defines a second angle in a range of 10 degrees to 50 degrees.

9. The system of claim 1, wherein a centerline length through the first bend, a section of the cannula extending between the first bend and the second bend, and the second bend combined may be in a range of 10 mm to 40 mm and/or other suitable ranges.

10. The system of claim 1, wherein the cannula is formed from a polymer material.

11. The system of claim 1, wherein the cannula is formed from a shape set nickel-titanium alloy defining the first bend and the second bend.

12. The system of claim 1, wherein the impeller assembly comprises an impeller and the impeller rotates in response to actuation of the motor to draw blood from the left ventricle into the cannula and through the impeller housing into the aorta.

13. A mechanical circulatory support system comprising:
a blood pump configured to pump blood from a left ventricle of a heart of a patient to an aorta of the patient;
a cannula extending distally from the blood pump, and
wherein the cannula comprises:
a first bend having a first angle in a plane and configured to direct the cannula through an aortic valve of the patient when the blood pump is pumping blood from the left ventricle to the aorta, and
a second bend having a second angle in the plane.

14. The system of claim 13, wherein the first bend and the second bend are configured to be located proximal of the aortic valve when the blood pump is pumping blood from the left ventricle to the aorta.

15. The system of claim 13, wherein the first angle is in a range of 10 degrees to 50 degrees and the second angle is in a range of 10 degrees to 50 degrees.

16. The system of claim 13, wherein a centerline length through the first bend, a section of the cannula extending between the first bend and the second bend, and the second bend combined may be in a range of 10 mm to 40 mm and/or other suitable ranges.

17. A method, comprising:
inserting a mechanical circulatory support system into a vasculature of a patient, the mechanical circulatory support system comprising:
a blood pump;
a cannula extending distally from the blood pump, the cannula having a first bend and a second bend located proximal of the first bend;
delivering the mechanical circulatory support system through the vasculature to a heart of the patient, wherein the blood pump and the cannula are delivered through the vasculature within a sheath; and
engaging the second bend with a wall of an aorta of the patient as the blood pump exits a distal end of the sheath.

18. The method of claim 17, further comprising:
delivering the cannula to a left ventricle of the heart of the patient while engaging the second bend with the wall of the aorta of the patient.

19. The method of claim 17, wherein the first bend is in a plane and the second bend is in the plane.

20. The method of claim 17, wherein the blood pump comprises an impeller assembly having an impeller housing and the second bend is proximate a distal end of the impeller housing.
